(19) United States
(12) Patent Application Publication
(10) Pub. No.: US 2025/0143182 A1
(43) Pub. Date: May 1, 2025

(54) ORGANIC MOLECULE FOR OPTOELECTRONIC DEVICE

(71) Applicant: Samsung Display Co., Ltd., Yongin-si, Gyeonggi-do (KR)

(72) Inventor: Michael DANZ, Eggenstein-Leopoldshafen (DE)

(21) Appl. No.: 18/835,320

(22) PCT Filed: Feb. 23, 2023
(86) PCT No.: PCT/KR2023/002624

§ 371 Date: Aug. 1, 2024

(30) Foreign Application Priority Data
Feb. 23, 2022 (EP) 22158231.5

(51) Int. Cl. H10K 85/60; C07D 209/88; C09K 11/02; C09K 11/06; H10K 50/11

(52) U.S. Cl. CPC H10K 85/6572; C07D 209/88; C09K 11/02; C09K 11/06; C09K 2211/1007; C09K 2211/1014; C09K 2211/1018; H10K 50/11

(57) ABSTRACT

The present disclosure relates to a pure organic molecule (free of a metal center) and use thereof as an emitter or absorber in an optoelectronic device. According to the present disclosure, the pure organic molecule includes a structure of Formula A.

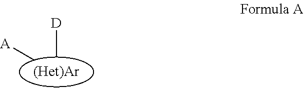

wherein
(Het)Ar=a conjugated organic group selected from the group consisting of unsubstituted and substituted aromatics, unsubstituted and substituted heteroaromatics, and conjugated double bonds fixed against cis-trans isomerization,
D=a chemically bonded donor group having electron donating properties,
A=a chemically bonded acceptor group having electron accepting properties, and
A and D are bonded to adjacent atoms of (Het)Ar.

### CROSS-REFERENCE TO RELATED APPLICATION

The present application is a U.S. National Phase Patent Application of International Patent Application Number PCT/KR2023/002624, filed on Feb. 23, 2023, which claims priority to and the benefit of European Patent Application Number 22158231.5, filed on Feb. 23, 2022, the entire content of each of which is incorporated herein by reference.

### TECHNICAL FIELD

The present disclosure relates to a pure organic molecule (dye) of Formula A, free of a metal center, and use thereof as an emitter in organic light-emitting diodes (OLEDs) and other optoelectronic components/devices.

### BACKGROUND ART

Recently, technologies based on organic light-emitting diodes (OLEDs) have introduced and settled in the field of display technology, and the initial commercial products based on these have emerged. In addition to screen technology, OLEDs are also suitable for flat lighting technology.

These devices are generally implemented as layered structures mainly consisting of organic materials. For better understanding, an example of a simplified structure is illustrated in FIG. 1. The key element of such a device (e.g., organic light-emitting device) is an emission layer in which a light-emitting molecule is generally embedded in a matrix. In the emission layer, negative charge carriers (electrons) and positive charge carriers (holes) meet and recombine to form so-called excitons (i.e., excited state). Energy contained in the excitons can be emitted in the form of light by the corresponding emitter, which is referred to as electroluminescence. An overview of the functions of OLEDs is described in, for example, Top. Curr. Chem., 2004, 241, 1, H. Yersin, and “Highly Efficient OLEDs with Phosphorescent Materials,” H Yersin; Wiley-VCH, Weinheim, Germany, 2018.

### DISCLOSURE

### Technical Problem

Since the first report related to OLEDs (Tang et al. Appl. Phys. Lett. 1987, 51, 913), the above technology has continued to be developed, especially in the field of emitter materials. The first materials based on pure organic molecules can convert up to 25% of excitons into light according to spin statistics, but when phosphorescent compounds are used, the fundamental spin statistics problem can be avoided. Thus, theoretically, all excitons can be converted into light. However, these materials have drawbacks, particularly in terms of cost and stability of materials used.

For triplet emitters suitable for triplet harvesting, transition metal complexes in which a metal is selected from the third-row transition metals are generally used. These are mainly very expensive precious metals such as iridium, platinum, or gold. (With regard to this, see the following: Top. Curr. Chem., 2004, 241, 1, H. Yersin, and Phys. Rev. B 1999, 60, 14422, M. A. Baldo, D. F. O'Brien, M. E. Thompson, S. R. Forrest).

### Technical Solution

Surprisingly, the above-described drawbacks can be considerably improved or solved by the present disclosure which uses an organic molecule (dye, emitter molecule) of Formula A.

The organic molecule according to the present disclosure includes a central basic unit in the form of a π-system (pi system), i.e., an aromatic (e.g., a heteroaromatic or an aromatic) which is substituted with a functional group having electron donating properties (donor) and a functional group having electron withdrawing properties (acceptor). Both groups are located on adjacent atoms of the central basic unit, i.e., at ortho-position relative to each other. Due to the ortho positioning of these groups (donor and acceptor), these groups have to rotate with respect to each other and thus be orthogonal to each other, resulting in electronic separation from each other. This separation may be understood, for example, by quantum mechanical calculations of highest occupied molecular orbital (HOMO) and lowest unoccupied molecular orbital (LUMO). Therefore, these two frontier orbitals should not overlap or should exhibit only minimal overlap. The π-system of the central basic unit may be mainly part of HOMO or LUMO.

Other than these groups (donor and acceptor), no group electronically affects the π-system (central basic unit). Permitted additional substituents may be alkyl groups or other groups that, for example, have a solubility effect.

The present disclosure provides an organic molecule comprising or consisting of a structure of Formula A which can be used in optoelectronic devices.

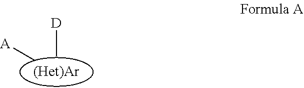

In formula A,

(Het)Ar=a conjugated organic group selected from the group consisting of unsubstituted and substituted aromatics, unsubstituted and substituted heteroaromatics, and conjugated double bonds fixed against cis-trans isomerization,

D=a chemically bonded donor group having electron donating properties, and

A=a chemically bonded acceptor group having electron accepting properties, wherein A and D are bonded to directly adjacent atoms of the conjugated organic group (Het)Ar.

In an embodiment of the present disclosure, the organic molecule has a value ΔE(S₁−T₁) between the lowest excited singlet (S₁) state and a triplet (T₁) state therebelow of less than 5000 cm⁻¹, less than 3000 cm⁻¹, or less than 2000 cm⁻¹.

In an embodiment of the present disclosure, the organic molecule has an emission lifetime of at most 50 μs, at most 30 μs, or at most 20 μs.

In another embodiment, the organic molecule has both the value ΔE(S₁−T₁) presented herein and the emission lifetime presented herein.

Examples of donors include aromatics and heteroaromatics that inherently exhibit electron donating properties and/or are substituted with electron donating groups, and other donors directly attached to the central π-system (aromatic or heteroaromatic) (i.e., central basic unit).

Donors may be:

—O(−), an —N(H)-alkyl group, an —N-(alkyl group)₂-NH₂, —OH, an —O-alkyl group, an —NH(CO)-alkyl group, an —O(CO)-alkyl group, an -alkyl group, a -phenyl group, and/or a —(CH)═C-(alkyl group)₂.

Examples of acceptors include aromatics and heteroaromatics that inherently exhibit electron accepting properties and/or are substituted with electron accepting groups, and other donors directly attached to the central π-system (aromatic or heteroaromatic).

Acceptors may be:
a halogen, —(CO)H, an —(CO)-alkyl group, an —(CO)O-alkyl group, —(CO)OH, —(CO)Cl, —CF₃, —CN, —S(O)₂OH, —NH₃(+), an —N(alkyl group)₃(+), and/or N(O)₂.

Optionally, the organic molecule may have at least one solubilizing group, for example, at least one alkyl group. The solubilizing group increases the solubility of the organic molecule in a given solvent.

In an embodiment, the substituent for increasing solubility (solubilizing group) may be selected from the group consisting of:
branched or unbranched or cyclic long alkyl chains with a length of C₁ to C₃₀;
branched or unbranched or cyclic alkoxy long chains with a length of C₁ to C₃₀;
branched or unbranched or cyclic long perfluoroalkyl chains with a length of C₁ to C₃₀; and
short-chain polyethers with a chain length of 3 to 50 repeating units.

In an embodiment of the present disclosure, the organic molecule has a phenyl ring (as illustrated in Formula B) as the central π-system.

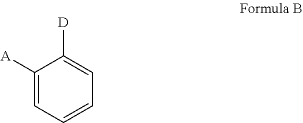

In another embodiment of the present disclosure, at least one selected from among the two substituents (i.e., donor and/or acceptor) of the central basic unit includes a heteroatom X, and is bonded to the central basic unit (π-system) via the heteroatom.

The heteroatom X is independently selected from the group consisting of N, S, P, O, and Se.

In another embodiment of the present disclosure, the two substituents (i.e., donor and acceptor) may each independently be bonded to the central basic unit (π-system) via the heteroatom X independently selected from the group consisting of N, S, P, O, and Se.

In another embodiment of the present disclosure, the organic molecule has a structure of Formula C:

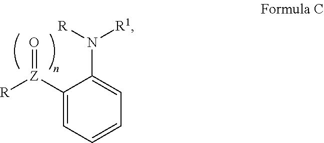

wherein
NRR¹ may be a donor (corresponding to D of Formula A or B),
R and R¹ may each independently be selected from
the group consisting of: hydrogen; a halogen; and a substituent bonded via a direct bond, or via oxygen- (—OR), nitrogen- (—NR₂), silicon- (—SiR₃), or sulfur- (—SR), and
the substituent may be selected from the group consisting of:
alkyl groups (including branched or cyclic), heteroalkyl groups, aryl groups, heteroaryl groups, alkenyl groups, and alkynyl groups;
alkyl groups (including branched or cyclic), heteroalkyl groups, aryl groups, heteroaryl groups, and alkenyl groups, each substituted with a substituent such as a halogen, deuterium, an alkyl group (including branched or cyclic), a heteroalkyl group, an aryl group, or a heteroaryl group; and
other commonly known donor and acceptor groups such as amines, alcohol and ether groups, carbonyl groups, carboxylates and esters thereof, nitrile groups, and CF₃ groups.

Also, the residues R and R¹ may optionally lead to or form a fused ring system.

“Oxidized” heteroatom Z(═O)ₙ may be an acceptor (corresponding to A of Formula A or B),
Z may be selected from the group consisting of S and P,
n may be 1 or 2, to which additional residue R is bonded,
the residue may each independently be selected from
the group consisting of: hydrogen; a halogen; and a substituent bonded via a direct bond, or via oxygen- (—OR), nitrogen- (—NR₂), silicon- (—SiR₃), or sulfur- (—SR), and
the substituent may be selected from the group consisting of:
alkyl groups (including branched or cyclic), heteroalkyl groups, aryl groups, heteroaryl groups, alkenyl groups, and alkynyl groups;
alkyl groups (including branched or cyclic), heteroalkyl groups, aryl groups, heteroaryl groups, and alkenyl groups, each substituted with a substituent such as a halogen, deuterium, an alkyl group (including branched or cyclic), a heteroalkyl group, an aryl group, or a heteroaryl group; and
other commonly known donor and acceptor groups such as amines, alcohol and ether groups, carbonyl groups, carboxylates and esters thereof, nitrile groups, and CF₃ groups.

Also, the residues R may optionally lead to or form a fused ring system.

Examples of these acceptors include SO₂R, SO₂(OR), S(O)R, P(O)RR′, P(O)(OR)(OR′), P(O)R(OR′), SeO₂R, and Se(O)R.

In another embodiment of the present disclosure, the organic molecule has a structure of Formula D:

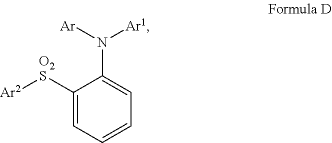

wherein
NArAr¹ may be a donor (corresponding to D of Formula A or B),
Ar and Ar¹ may each independently be aryl or heteroaryl to which additional residue R is optionally bonded,
the residue may each independently be selected from
the group consisting of: hydrogen; a halogen; and a substituent bonded via a direct bond, or via oxygen- (—OR), nitrogen- (—NR₂), silicon- (—SiR₃), or sulfur- (—SR), and
the substituent may be selected from the group consisting of:
alkyl groups (including branched or cyclic), heteroalkyl groups, aryl groups, heteroaryl groups, alkenyl groups, and alkynyl groups;
alkyl groups (including branched or cyclic), heteroalkyl groups, aryl groups, heteroaryl groups, and alkenyl groups, each substituted with a substituent such as a halogen, deuterium, an alkyl group (including branched or cyclic), a heteroalkyl group, an aryl group, or a heteroaryl group; and
other commonly known donor and acceptor groups such as amines, alcohol and ether groups, carbonyl groups, carboxylates and esters thereof, nitrile groups, and CF₃ groups.

Also, the residue R may optionally lead to or form a fused ring system.

Ar and Ar¹ may also be fused with each other or linked to each other via unit E (e.g., carbazole or phenoxazine),
the unit E is a direct bond, or
an organic bridge that is: a substituted or unsubstituted alkylene group, a substituted or unsubstituted alkenylene group, a substituted or unsubstituted alkynylene group, a substituted or unsubstituted arylene group, or a combination thereof;
—O—, —NR—, —SiR₂—, —S—, —S(O)—, —S(O)₂—, an alkyl group interrupted by O (including branched or cyclic), a heteroalkyl group, an aryl group, a heteroaryl group, an alkenyl group, or a phenyl group; or
a substituted phenyl unit,
wherein R may each independently be selected from
the group consisting of: hydrogen; a halogen; and a substituent bonded via a direct bond, or via oxygen- (—OR), nitrogen- (—NR₂), silicon- (—SiR₃), or sulfur- (—SR), and
the substituent may be selected from the group consisting of:
alkyl groups (including branched or cyclic), heteroalkyl groups, aryl groups, heteroaryl groups, alkenyl groups, and alkynyl groups;
alkyl groups (including branched or cyclic), heteroalkyl groups, aryl groups, heteroaryl groups, and alkenyl groups, each substituted with a substituent such as a halogen, deuterium, an alkyl group (including branched or cyclic), a heteroalkyl group, an aryl group, or a heteroaryl group; and
other commonly known donor and acceptor groups such as amines, alcohol and ether groups, carbonyl groups, carboxylates and esters thereof, nitrile groups, and/or CF₃ groups.

Also, the residue R may optionally lead to or form a fused ring system.

SO₂Ar² is an acceptor (corresponding to A of Formula A or B),
wherein Ar² may be aryl or heteroaryl to which additional residue R is optionally bonded,
the residue may each independently be selected from
the group consisting of: hydrogen; a halogen; and a substituent bonded via a direct bond, or via oxygen- (—OR), nitrogen- (—NR₂), silicon- (—SiR₃), or sulfur- (—SR), and
the substituent may be selected from the group consisting of:
alkyl groups (including branched or cyclic), heteroalkyl groups, aryl groups, heteroaryl groups, alkenyl groups, and alkynyl groups;
alkyl groups (including branched or cyclic), heteroalkyl groups, aryl groups, heteroaryl groups, and alkenyl groups, each substituted with a substituent such as a halogen, deuterium, an alkyl group (including branched or cyclic), a heteroalkyl group, an aryl group, or a heteroaryl group; and
other commonly known donor and acceptor groups such as amines, alcohol and ether groups, carbonyl groups, carboxylates and esters thereof, nitrile groups, and CF₃ groups.

Also, the residue R may optionally lead to or form a fused ring system.

Ar² may also be fused with other aryl and heteroaryl groups to form a larger aromatic system (e.g., phenyl, naphthyl, anthracenyl, tolyl, or the like).

In another embodiment of the present disclosure, the organic molecule has a structure of Formula E:

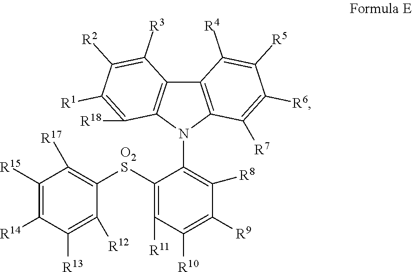

wherein
R¹ to R¹⁸ are each independently selected from the group consisting of:
hydrogen; a halogen; and a substituent bonded via a direct bond, or via oxygen- (—OR), nitrogen- (—NR₂), silicon- (—SiR₃), or sulfur- (—SR) (wherein the residue R is as defined above for R in Formula D), and
the substituent may be selected from the group consisting of:
alkyl groups (including branched or cyclic), heteroalkyl groups, aryl groups, heteroaryl groups, alkenyl groups, and alkynyl groups;
alkyl groups (including branched or cyclic), heteroalkyl groups, aryl groups, heteroaryl groups, and alkenyl groups, each substituted with a substituent such as a halogen, deuterium, an alkyl group (including branched or cyclic), a heteroalkyl group, an aryl group, or a heteroaryl group; and
other commonly known donor and acceptor groups, for example, amines, alcohol and ether groups, carbonyl groups, carboxylates and esters thereof, nitrile groups, and CF₃ groups which are optionally further substituted and/or fused.

Also, the residues R¹ to R¹⁸ may optionally and independently lead to or form a condensed ring system.

According to another aspect, the present disclosure relates to a method of preparing the above-described organic molecule. In an embodiment, the method comprises the step of substituting at least one selected from among the three groups, i.e., (Het)Ar, A, and/or D (of Formula A or B), with at least one substituent for increasing solubility, wherein the substituent may be selected from the group consisting of:
long-chain, branched or unbranched or cyclic alkyl chains with a length of C₁ to C₃₀;
long-chain, branched or unbranched or cyclic alkoxy chains with a length of C₁ to C₃₀;
branched or unbranched or cyclic perfluoroalkyl chains with a length of C₁ to C₃₀; and
short-chain polyethers with a chain length of 3 to 50 repeating units.

According to another aspect, the present disclosure relates to use of the organic molecule as described above as an emitter or absorber in an optoelectronic device, and the optoelectronic device may be selected from the group consisting of:
organic light-emitting devices (OLEDs),
light-emitting electrochemical cells,
OLED sensors, especially in gas and vapor sensors not hermetically externally shielded,
organic solar cells,
organic field-effect transistors,
organic lasers, and
down-conversion elements.

The proportion of the organic molecule as an emitter or an absorber is in a range of 1% to 99% by weight based on a total weight of 100% by weight of a function layer including the organic molecule in the optoelectronic device. In an embodiment of the present disclosure, the proportion of the organic molecule as an emitter or an absorber may be 100% by weight based on a total weight of the function layer.

In an embodiment, the proportion of the organic molecule as an emitter in an optical light-emitting device, particularly OLED, is in a range of 5% to 80% by weight based on a total weight of 100% by weight of a function layer including the organic molecule in the optical light-emitting device.

According to another aspect, the present disclosure relates to an optoelectronic device including the organic molecule as described above. In particular, the optoelectronic device may be selected from the group consisting of organic light-emitting devices, organic diodes, organic solar cells, organic transistors, organic light-emitting diodes, light-emitting electrochemical cells, organic field-effect transistors, and organic lasers.

According to another aspect, the present disclosure relates to a method of producing an optoelectronic device using the organic molecule as described above.

Part of this method may be a step of applying the organic molecule according to the present disclosure to a carrier, and the application may optionally be performed by wet-chemical, by colloidal suspension, or by sublimation.

According to another aspect, the present disclosure relates to a method of modifying the emission and/or absorption properties of an electronic device, the method including a step of introducing the organic molecule according to the present disclosure into a matrix material for conducting electrons or holes in an optoelectronic device.

According to another aspect, the present disclosure relates to use of the organic molecule as described above in an optoelectronic device, and relates to an optoelectronic device for converting (so-called down conversion) UV radiation or blue light into visible light, particularly green light, yellow light, or red light.

### DESCRIPTION OF DRAWINGS

The drawings are as follows:

FIG. 1 schematically illustrates an example of the structure of an organic light emitting diode (OLED) (the device includes at least one anode, a cathode, and an emission layer, and the given thicknesses of layers are provided for illustrative purposes only).

FIG. 2 is an HOMO/LUMO image of organic molecule 1.

FIG. 3 is an HOMO/LUMO image of organic molecule 2.

FIG. 4 illustrates the emission spectrum of organic molecule 1 dissolved in toluene (arbitrary units of intensity (a.u.) versus wavelength length (nm)).

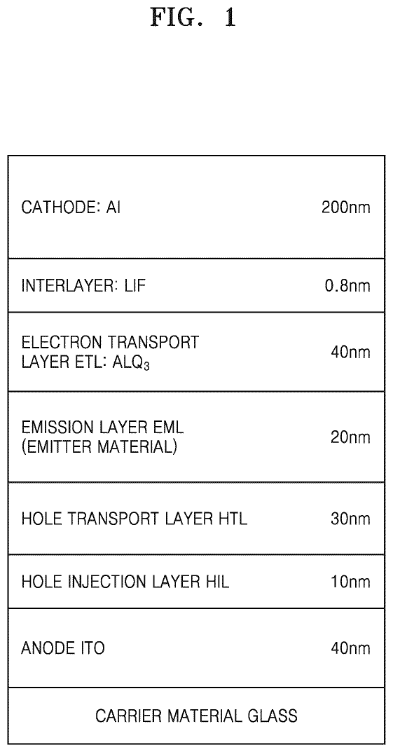

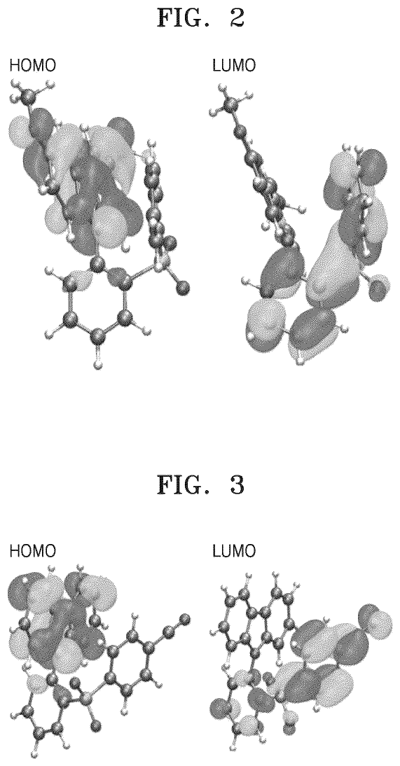

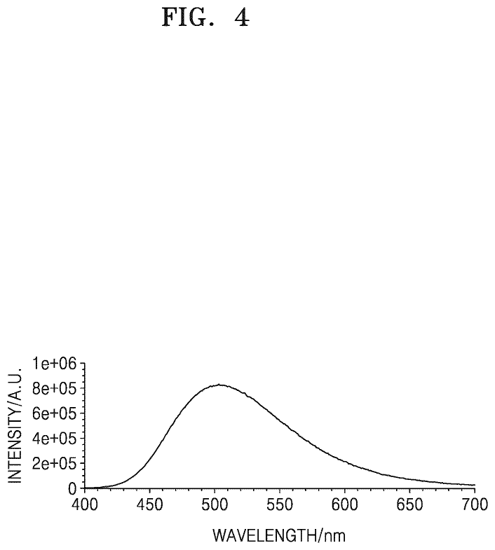

### MODE FOR INVENTION

### Examples

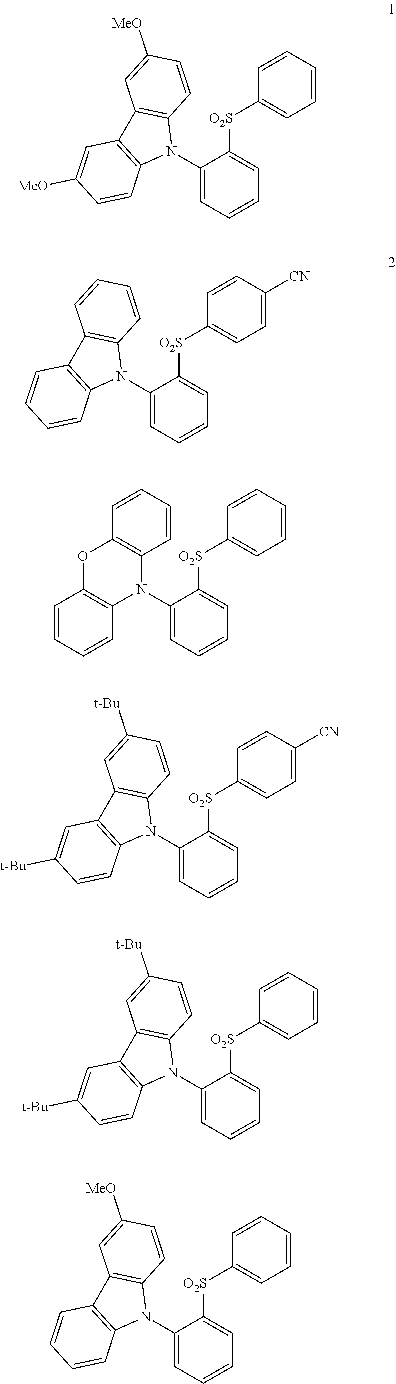 [SMILES: COc1ccc2c(c1)c1cc(OC)ccc1n2-c1ccccc1S(=O)(=O)c1ccccc1 ; N#Cc1ccc(S(=O)(=O)c2ccccc2-n2c3ccccc3c3ccccc32)cc1 ; O=S(=O)(c1ccccc1)c1ccccc1N1c2ccccc2Oc2ccccc21 ; CC(C)(C)c1ccc2c(c1)c1cc(C(C)(C)C)ccc1n2-c1ccccc1S(=O)(=O)c1ccc(C#N)cc1 ; CC(C)(C)c1ccc2c(c1)c1cc(C(C)(C)C)ccc1n2-c1ccccc1S(=O)(=O)c1ccccc1 ; COc1ccc2c(c1)c1ccccc1n2-c1ccccc1S(=O)(=O)c1ccccc1]

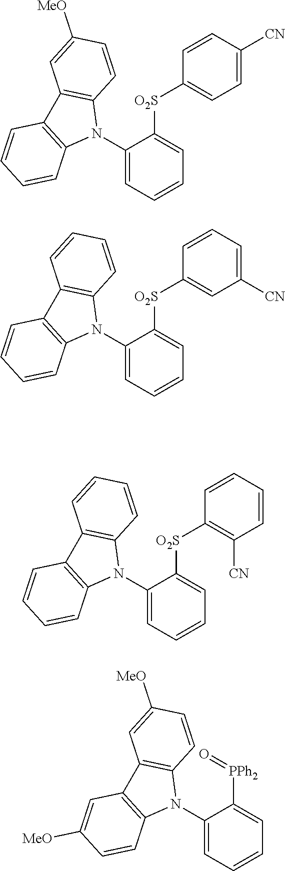

### Synthesis Method

### Synthesis of 1

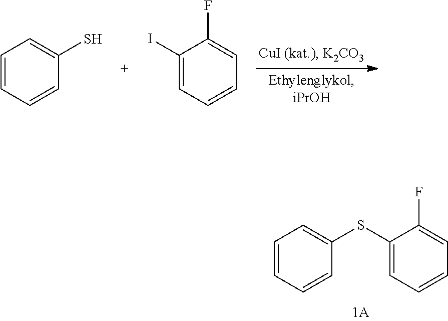 [SMILES: Sc1ccccc1 ; Fc1ccccc1I ; Fc1ccccc1Sc1ccccc1]

Copper(I) iodide (479 mg, 2.5 mmol) and potassium carbonate (13.8 g, 100 mmol) were placed in a 100 mL two-necked flask with a reflux condenser and evacuated for 5 minutes. Under atmosphere including nitrogen, 50 mL of 2-propanol, 1-fluoro-2-iodobenzene (12.2 g, 55 mmol), thiophenol (5.1 mL, 50 mmol), and ethylene glycol (5.6 mL, 100 mmol) were added. The mixture was stirred at 90° C. for 1 day. The reaction solution was filtered, and the residue was washed with 100 mL of water and 100 mL of EtOAc. After phase separation, the aqueous phase was extracted again with 100 mL of EtOAc. The combined organic phase was washed with a saturated NaCl solution and dried over MgSO₄. The solvent was removed under reduced pressure on a rotary evaporator and the residue was filtered through a small amount of silica gel (eluent: CH₂Cl₂). The solvent was removed again under reduced pressure on a rotary evaporator. 9.8 g of diarylsulfide 1A was obtained as colorless oil that contained approximately 10% 1-fluoro-2-iodobenzene but could react further without further purification.

¹H-NMR (500 MHz, CDCl₃): δ=7.41-7.37 (m, 2H), 7.37-7.32 (m, 2H), 7.32-7.27 (m, 3H), 7.16-7.08 (m, 2H).

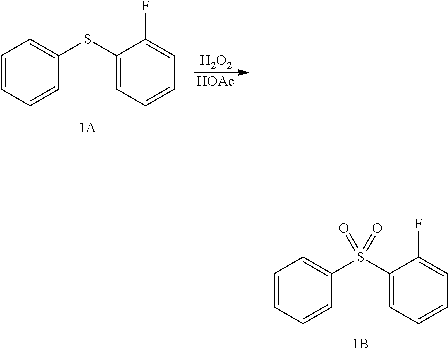

Diarylsulfide 1A (purity 90%, 6.81 g, 30 mmol) was dissolved in 150 mL of glacial acetic acid and treated with 15 mL of H₂O₂ (30% in water). The mixture was stirred at 100° C. for 2 hours, and then left at room temperature overnight. The reaction solution was poured into 800 mL of water, causing the product to precipitate as a white solid. The solid was filtered and washed with water. The resulting filtrate was dried under vacuum at 50° C. overnight. 5.8 g of diarylsulfone 1B was obtained as a white solid.

¹H-NMR (500 MHz, CDCl₃): δ=8.11 (td, J=7.5, 1.8 Hz, 1H), 8.05-7.98 (m, 2H), 7.65-7.55 (m, 2H), 7.53 (t, J=7.8 Hz, 2H), 7.32 (t, J=7.7 Hz, 1H), 7.14-7.07 (m, 1H).

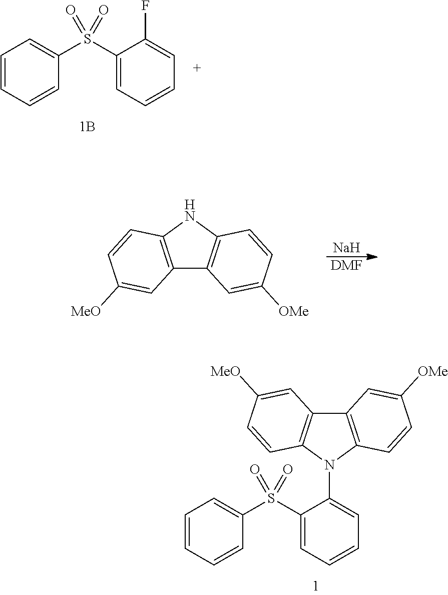

3,6-dimethoxycarbazole (696 mg, 3 mmol) was dissolved in 10 mL of dry dimethylformamide (DMF), and NaH (60% in mineral oil, 240 mg, 6 mmol) was added thereto. After stirring for 30 minutes, diarylsulfone 1B dissolved in 10 mL of DMF was added and the mixture was stirred at 100° C. for 2 hours. The reaction solution was poured into 200 mL of ice water. After being left overnight, the solid was filtered and washed with some water. The solid was purified by column chromatography (eluent: CH₂Cl₂/cyclohexane). 350 mg of product 1 was obtained, which could further be purified by recrystallization from EtOH.

¹H-NMR (500 MHz, CDCl₃): δ=8.61 (dd, J=7.8, 1.8 Hz, 1H), 7.74 (dtd, J=18.7, 7.5, 1.6 Hz, 2H), 7.44 (d, J=2.5 Hz, 2H), 7.13 (ddt, J=5.5, 4.0, 2.2 Hz, 2H), 7.11-7.07 (m, 2H), 6.88 (t, J=7.9 Hz, 2H), 6.76 (dd, J=8.8, 2.5 Hz, 2H), 6.41 (d, J=8.8 Hz, 2H), 3.92 (s, 6H).

¹³C-NMR (125 MHz, CDCl₃): δ=154.3, 142.7, 139.4, 138.2, 137.1, 135.3, 132.8, 132.8, 130.0, 129.7, 128.4, 127.9, 123.9, 115.0, 111.6, 102.7, 56.3.

### Synthesis of 2

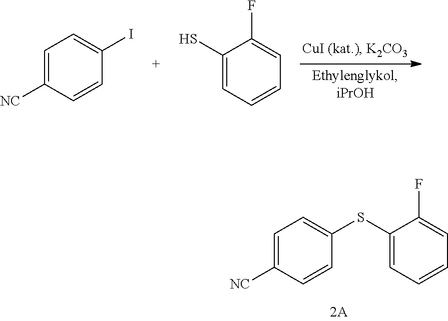 [SMILES: N#Cc1ccc(I)cc1 ; Fc1ccccc1S ; N#Cc1ccc(Sc2ccccc2F)cc1]

4-iodobenzonitrile (11.5 g, 500 mmol), copper(I) iodide (957 mg, 5 mmol), and potassium carbonate (13.8 g, 100 mmol) were placed in a 250 mL two-necked flask with a reflux condenser and evacuated for 5 minutes. Under nitrogen atmosphere, 100 mL of 2-propanol (iPrOH), 1-fluoro-2-thiobenzene (6.41 g, 50 mmol), and ethylene glycol (5.6 mL, 100 mmol) were added. The mixture was stirred at 90° C. for 1 day. The reaction solution was filtered and the residue was washed with 300 mL of EtOAc. The solvent was removed under reduced pressure on a rotary evaporator, and the residue was dissolved in 100 mL of EtOAc and 100 mL of water. The organic phase was washed once more with 100 mL water, followed by washing with 50 mL of a saturated NaCl solution and dried over MgSO₄. The solvent was removed under reduced pressure on a rotary evaporator, and the residue was filtered through a small amount of silica gel (eluent: CH₂Cl₂). The solvent was removed again under reduced pressure on a rotary evaporator. 10.5 g of diarylsulfide 2A was obtained as a white solid, which contains about 5% 4-iodobenzonitrile but can further be converted without further purification.

¹H-NMR (500 MHz, CDCl₃): δ=7.57-7.43 (m, 4H), 7.25-7.19 (m, 2H), 7.19-7.15 (m, 2H).

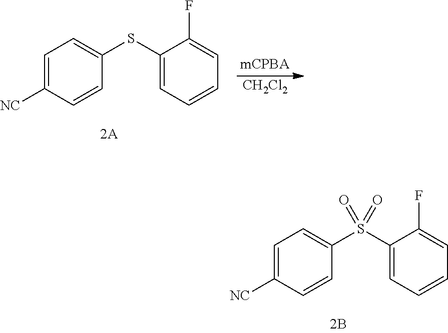

Diarylsulfide 2A (purity 95%, 5.31 g, 22 mmol) was dissolved in 150 mL of CH₂Cl₂. Meta-chloroperbenzoic acid (mCPBA) (70%, 16.3 g, 66 mmol) was added over approximately 10 minutes under ice cooling. Subsequently, the mixture was stirred at room temperature for 16 hours. The reaction solution was washed with 100 mL of a semi-concentrated Na₂CO₃ solution, and then washed with 50 mL of a saturated NaCl solution and dried over MgSO₄. The solvent was removed under reduced pressure on a rotary evaporator and the residue was filtered through a small amount of silica gel (eluent: CH₂Cl₂). The solvent was removed again under reduced pressure on a rotary evaporator. 5.2 g of diarylsulfone 2B was obtained as a light yellow solid.

1H-NMR (500 MHz, CDCl₃): δ=8.17-8.08 (m, 3H), 7.85-7.81 (m, 2H), 7.64 (tdd, J=7.4, 4.9, 1.8 Hz, 1H), 7.37 (t, J=7.7 Hz, 1H), 7.18-7.10 (m, 1H).

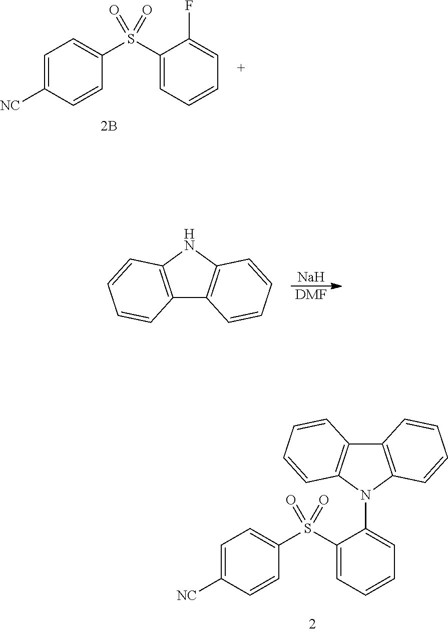

Carbazole (2.51 g, 15 mmol) was dissolved in 50 mL of dry DMF, and NaH (60% in mineral oil, 1.20 g, 30 mmol) was added thereto. After stirring for 15 minutes, diarylsulfone 2B dissolved in 50 mL of DMF was added and the mixture was stirred at 100° C. for 2 hours. The reaction solution was poured onto 800 mL of ice. After being left overnight, the solid was filtered and washed with water. The solid was purified by column chromatography (eluent: CH₂Cl₂). 3.14 g of product 2 was obtained as a white solid.

¹H-NMR (500 MHz, CDCl₃): δ=8.66-8.61 (m, 1H), 8.03 (d, J=7.7 Hz, 2H), 7.89-7.80 (m, 2H), 7.30-7.23 (m, 5H), 7.19 (ddd, J=8.2, 7.1, 1.2 Hz, 2H), 7.11-7.01 (m, 4H), 6.53 (d, J=8.0 Hz, 2H).

¹³C-NMR (125 MHz, CDCl₃): δ=142.8, 141.9, 141.8, 136.1, 136.0, 133.0, 132.0, 130.3, 130.2, 128.1, 126.1, 123.3, 120.8, 120.1, 117.1, 116.2, 110.4.

1-20. (canceled)

21. An organic molecule comprising a structure of Formula A: 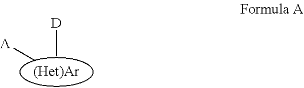
wherein,
(Het)Ar is a conjugated organic group selected from the group consisting of unsubstituted and substituted aromatics, unsubstituted and substituted heteroaromatics, and conjugated double bonds fixed against cis-trans isomerization,
D is a chemically bonded donor group having electron donating properties,
A is a chemically bonded acceptor group having electron accepting properties, and
A and D are bonded to directly adjacent atoms of the conjugated organic group (Het)Ar.

22. The organic molecule of claim 21, wherein the organic molecule has
a value ΔE(S₁−T₁) between a lowest excited singlet (S₁) state and a triplet (T₁) state of less than 5000 cm⁻¹, the triplet (T₁) state having an energy less than that of the lowest excited singlet (S₁) state, and/or
an emission lifetime of 50 μs or less.

23. The organic molecule of claim 21, wherein (Het)Ar, A, and/or D has at least one substituent being to increase a solubility of the organic molecule in an organic solvent, and wherein the at least one substituent is selected from the group consisting of:
branched or unbranched or cyclic alkyl chains with a length of C₁ to C₃₀;
branched or unbranched or cyclic alkoxy chains with a length of C₁ to C₃₀;
branched or unbranched or cyclic perfluoroalkyl chains with a length of C₁ to C₃₀; and
short-chain polyethers with a chain length of 3 to 50 repeating units.

24. The organic molecule of claim 21, wherein (Het)Ar is phenyl.

25. The organic molecule of claim 21, wherein at least one selected from among the two substituents A and D has a heteroatom X through which the substituent is covalently bonded to (Het)Ar.

26. The organic molecule of claim 25, wherein the heteroatom X is selected from the group consisting of N, S, P, O, and Se.

27. The organic molecule of claim 21, wherein the organic molecule has a structure of Formula C: 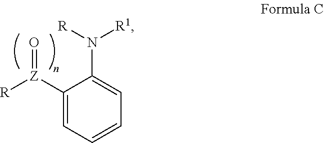
wherein,
NRR¹ is a donor,
R and R¹ are each independently selected from
the group consisting of: hydrogen; a halogen; a substituent bonded via a direct bond; a substituent bonded via oxygen- (—OR); a substituent bonded via nitrogen- (—NR₂); a substituent bonded via silicon- (—SiR₃); and a substituent bonded via sulfur- (—SR),
the substituent being independently selected from the group consisting of:
alkyl groups, cycloalkyl groups, heteroalkyl groups, aryl groups, heteroaryl groups, alkenyl groups, and alkynyl groups;
alkyl groups, cycloalkyl groups, heteroalkyl groups, aryl groups, heteroaryl groups, and alkenyl groups, each substituted with at least one substituent selected from among a halogen, deuterium, an alkyl group, a cycloalkyl group, a heteroalkyl group, an aryl group, and a heteroaryl group; and
amines, alcohol and ether groups, carbonyl groups, carboxylates and esters thereof, nitrile groups, and CF₃ groups,
R and R¹ optionally form a fused ring system,
heteroatom Z(═O)ₙ moiety is an acceptor and corresponds to A of Formula A,
n is 1 or 2, and
Z is selected from the group consisting of S and P, to which additional residue R is bonded,
the additional residue R being each independently selected from
the group consisting of: hydrogen; a halogen; a substituent bonded via a direct bond; a substituent bonded via oxygen- (—OR); a substituent bonded via nitrogen- (—NR₂); a substituent bonded via silicon- (—SiR₃); and a substituent bonded via sulfur- (—SR),
the substituent being independently selected from the group consisting of:
alkyl groups, cycloalkyl groups, heteroalkyl groups, aryl groups, heteroaryl groups, alkenyl groups, and alkynyl groups;
alkyl groups, cycloalkyl groups, heteroalkyl groups, aryl groups, heteroaryl groups, and alkenyl groups, each substituted with at least one substituent selected from among a halogen, deuterium, an alkyl groups including branched or cyclic, a heteroalkyl group, an aryl group, and a heteroaryl group; and
amines, alcohol and ether groups, carbonyl groups, carboxylates and esters thereof, nitrile groups, and CF₃ groups, and
the additional residues R optionally forming a fused ring system.

28. The organic molecule of claim 21, wherein the organic molecule has a structure of Formula D: 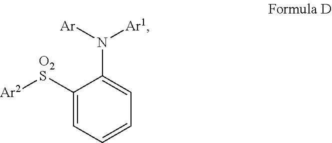
wherein,
NArAr¹ is a donor and corresponds to D of Formula A,
Ar and Ar¹ are each independently aryl or heteroaryl to which an additional residue R is optionally bonded,
the additional residue R is independently selected from
the group consisting of: hydrogen; a halogen; a substituent bonded via a direct bond; a substituent bonded via oxygen- (—OR); a substituent bonded via nitrogen-(—NR₂); a substituent bonded via silicon- (—SiR₃); and a substituent bonded via sulfur- (—SR),
the substituent being independently selected from the group consisting of:
alkyl groups, cycloalkyl groups, heteroalkyl groups, aryl groups, heteroaryl groups, alkenyl groups, and alkynyl groups;
alkyl groups, cycloalkyl groups, heteroalkyl groups, aryl groups, heteroaryl groups, and alkenyl groups, each substituted with at least one substituent selected from among a halogen, deuterium, an alkyl group, a cycloalkyl group, a heteroalkyl group, an aryl group, and a heteroaryl group; and
amines, alcohol and ether groups, carbonyl groups, carboxylates and esters thereof, nitrile groups, and CF₃ groups,
the additional residues R optionally forms a fused ring system,
Ar and Ar¹ are optionally fused with each other or linked to each other via unit E,
the unit E is
a direct bond; or
an organic bridge that is: a substituted or unsubstituted alkylene group, a substituted or unsubstituted alkenylene group, a substituted or unsubstituted alkynylene group, a substituted or unsubstituted arylene group, or a combination thereof; or
—O—, —NR—, —SiR₂— —S—, —S(O)—, —S(O)₂—, an alkyl group interrupted by O, a cycloalkyl group interrupted by O, a heteroalkyl group, an aryl group, a heteroaryl group, an alkenyl group, or a phenyl group; or
a substituted phenyl unit,
wherein each substituent R is independently selected from
the group consisting of: hydrogen; a halogen; a substituent bonded via a direct bond; a substituent bonded via oxygen- (—OR); a substituent bonded via nitrogen- (—NR₂); a substituent bonded via silicon- (—SiR₃); and a substituent bonded via sulfur- (—SR),
the substituent being independently selected from the group consisting of:
alkyl groups, cycloalkyl groups, heteroalkyl groups, aryl groups, heteroaryl groups, alkenyl groups, and alkynyl groups;
alkyl groups, cycloalkyl groups, heteroalkyl groups, aryl groups, heteroaryl groups, and alkenyl groups, each substituted with at least one substituent selected from among a halogen, deuterium, an alkyl group, a cycloalkyl group, a heteroalkyl group, an aryl group, and a heteroaryl group; and
amines, alcohol and ether groups, carbonyl groups, carboxylates and esters thereof, nitrile groups, and CF₃ groups,
the substituents R optionally form a fused ring system,
SO₂Ar² is an acceptor and corresponds to A of Formula A,
wherein Ar² is aryl or heteroaryl to which an additional residue R is optionally bonded,
the additional residue R is independently selected from
the group consisting of: hydrogen; a halogen; a substituent bonded via a direct bond; a substituent bonded via oxygen- (—OR); a substituent bonded via nitrogen-(—NR₂); a substituent bonded via silicon- (—SiR₃); and a substituent bonded via sulfur- (—SR),
the substituent being independently selected from the group consisting of:
alkyl groups, cycloalkyl groups, heteroalkyl groups, aryl groups, heteroaryl groups, alkenyl groups, and alkynyl groups;
alkyl groups, cycloalkyl groups, heteroalkyl groups, aryl groups, heteroaryl groups, and alkenyl groups, each substituted with at least one substituent selected from among a halogen, deuterium, an alkyl group, a cycloalkyl group, a heteroalkyl group, an aryl group, or a heteroaryl group; and
amines, alcohol and ether groups, carbonyl groups, carboxylates and esters thereof, nitrile groups, and CF₃ groups,
the additional residues R optionally forming a fused ring system, and
Ar is optionally fused with other aryl groups and/or heteroaryl groups to form a larger aromatic system.

29. The organic molecule of claim 21, wherein the organic molecule has a structure of Formula E: 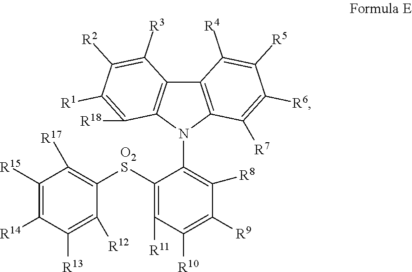
wherein,
R¹ to R¹⁸ are each independently selected from the group consisting of:
hydrogen; a halogen; a substituent bonded via a direct bond; a substituent bonded via oxygen- (—OR); a substituent bonded via nitrogen- (—NR₂); a substituent bonded via silicon- (—SiR₃); and a substituent bonded via sulfur (—SR),
the substituent being selected from the group consisting of:
alkyl groups, cycloalkyl groups, heteroalkyl groups, aryl groups, heteroaryl groups, alkenyl groups, and alkynyl groups;
alkyl groups, cycloalkyl groups, heteroalkyl groups, aryl groups, heteroaryl groups, and alkenyl groups, each substituted with at least one substituent independently selected from among a halogen, deuterium, an alkyl group, a cycloalkyl group, a heteroalkyl group, an aryl group, or a heteroaryl group; and
amines, alcohol and ether groups, carbonyl groups, carboxylates and esters thereof, nitrile groups, and CF₃ groups which are optionally further substituted and/or fused, and
R¹ to R¹⁸ optionally form a condensed ring system.

30. A method of preparing the organic molecule according to claim 21, the method comprising substituting at least one of (Het)Ar, A, or D with at least one substituent being to increase solubility, wherein the at least one substituent is selected from the group consisting of:
branched or unbranched or cyclic alkyl chains with a length of C₁ to C₃₀;
branched or unbranched or cyclic alkoxy chains with a length of C₁ to C₃₀;
branched or unbranched or cyclic perfluoroalkyl chains with a length of C₁ to C₃₀; and
short-chain polyethers with a chain length of 3 to 50 repeating units.

31. An optoelectronic device, comprising the organic molecule according to claim 21 as an emitter or an absorber.

32. The optoelectronic device of claim 31, wherein the optoelectronic device is at least one selected from the group consisting of:
organic light-emitting devices (OLEDs),
light-emitting electrochemical cells,
OLED sensors,
organic solar cells,
organic field-effect transistors,
organic lasers,
down-conversion elements, and
combinations thereof.

33. The optoelectronic device of claim 32, wherein a proportion of the organic molecule as an emitter or an absorber is in a range of 1% to 99% or 100% by weight based on a total weight of 100% by weight of a function layer comprising the organic molecule in the optoelectronic device.

34. The optoelectronic device of claim 32, wherein a proportion of the organic molecule as an emitter is in a range of 5% to 80% by weight based on a total weight of 100% by weight of a function layer comprising the organic molecule in the optoelectronic device.

35. The optoelectronic device of claim 34, wherein the optoelectronic device is an organic light emitting device.

36. A method of producing an optoelectronic device, the method comprising applying the organic molecule according to claim 21 to a carrier.

37. The method of claim 36, wherein the applying of the organic molecule to the carrier is performed by wet-chemical, by colloidal suspension, or by sublimation.

38. A method of modifying emission and/or absorption characteristics of an optoelectronic device, the method comprising introducing the organic molecule according to claim 21 into a matrix material being to conduct electrons or holes in the optoelectronic device.

39. A method, comprising converting UV radiation or blue light into visible light by utilizing the organic molecule according to claim 21 in an optoelectronic device.

40. The method of claim 39, wherein the visible light is green light, yellow light, or red light.
